(19) United States
(12) Patent Application Publication
(10) Pub. No.: US 2026/0232465 A1
(43) Pub. Date: Aug. 13, 2026

(54) DELIVERY SYSTEM FOR A SELF-EXPANDABLE BRAIDED STENT

(71) Applicant: MERIL LIFE SCIENCES PVT LTD, Vapi (IN)

(72) Inventors: PRAMOD KUMAR MINOCHA, Vapi (IN); DEVESHKUMAR MAHENDRALAL KOTHWALA, Surat (IN); NIRAVKUMAR MAHESHBHAI RANA, Valsad (IN); RIPALKUMAR BHARATBHAI PATEL, Valsad (IN)

(73) Assignee: MERIL LIFE SCIENCES PVT LTD, Vapi, GJ (IN)

(21) Appl. No.: 19/153,864

(22) PCT Filed: Jan. 17, 2024
(86) PCT No.: PCT/IN2024/050044

§ 371 Date: Aug. 5, 2025

(30) Foreign Application Priority Data
Feb. 6, 2023 (IN) 202321007368

(51) Int. Cl. A61F 2/95; A61F 2/90; A61F 2/966

(52) U.S. Cl. CPC A61F 2/9517; A61F 2/90; A61F 2/966; A61F 2002/9505

(57) ABSTRACT

A delivery system for delivering a self-expandable braided stent is disclosed. The delivery system includes a handle that contains a roller assembly. The roller assembly includes a roller, a base plate, a connecting shaft and a slider block. The slider block is supported on an inner tube. The delivery system includes a hypotube that is fixedly coupled to the slider block and co-axially receives the inner tube. The stent is mounted on the hypotube in a crimped state. An outer sheath is provided over the hypotube to cover the stent. On rotation of the roller, the slider block enables the forward and backward movement of the hypotube along the longitudinal axis ‘x’ over the inner tube relative to the outer sheath.

### FIELD OF INVENTION

The present invention relates to a delivery system. More specifically, the present invention relates to a delivery system for delivery of self-expandable braided stents at a target location.

### BACKGROUND OF THE INVENTION

A self-expanding stent is widely used in body vessels, ducts, or other body lumens to restore normal blood flow. The introduction and placement of a self-expanding stent is facilitated using a corresponding stent delivery system. The self-expanding stent is mounted on the delivery system in a crimped state retained by a sheath. Once the delivery system along with the stent, reaches a target location, the sheath is withdrawn due to which the stent is uncovered thereby expanding the stent radially. The expanded stent exerts a radial force at the target location to re-establish patency of a body lumen or duct.

The self-expanding stent is generally made by way of braiding or laser cutting. The conventional delivery systems for deploying self-expandable braided stents mostly work on a switch and lock mediated push-pull mechanism. For example, the prior art U.S. Pat. No. 9,023,095B2 discloses a delivery system for self-expandable stents based on a sliding mechanism having components such as a handle, a driver assembly, a pusher lock, a deployment lock, and a switch. The switch is connected with an inner lumen that slides over a supporting tube. In this delivery system, the operator has to be very careful during the deployment/delivery of the stent, as the operator has to push the stent a number of times to deliver the stent at a particular location. Also, the operator has to keep in mind that the deployment lock has to be unlocked for the final push to deliver/deploy the stent fully at a targeted location. Hence, the delivery system disclosed in the said patent publication is very complex and inconvenient to use for the operator thereby increasing the chances of inaccurate deployment of the stent and rendering the delivery system and mechanism thereof error-prone. Also, the delivery system is time-intensive i.e., requires long procedural time for deployment of a stent.

Further, owing to the increased procedural time in the conventional delivery system, targeting long lesions is a major challenge. The intervention techniques to treat long lesions include the use of multiple overlapping stents or the use of a single long stent. However, given that the conventional delivery system is already associated with increased procedural time, the procedural time for deploying a long stent is increased multi-fold thereby simultaneously enhancing the risk of stent migration, vessel injury, unnecessary stent stacking, etc.

Thus, in order to overcome the drawbacks of the conventional delivery systems, there is a need to devise a delivery system for a braided self-expandable stent which is operator-friendly, easy to operate and delivers a stent (including long stents) in a controlled manner in less procedural time.

### SUMMARY OF THE INVENTION

The present invention relates to a delivery system for delivering a self-expandable braided stent. The delivery system includes a handle that is disposed towards a proximal end of the delivery system. The handle encloses a roller assembly having a roller that is capable of rotating in a clockwise and an anti-clockwise direction. The roller assembly additionally includes a base plate, a connecting shaft and a slider block. The slider block is supported on an inner tube.

The delivery system also includes a hypotube having a proximal end and a distal end. The proximal end is coupled to the slider block of the roller assembly. The hypotube is provided with a stent driver that is disposed towards the distal end. The stent driver is disposed at a pre-defined distance from a distal end of the delivery system. The hypotube is provided with a soft tip at its distal end. The hypotube is configured to mount a self-expandable braided stent adjacent to the soft tip.

An outer sheath is coupled at a distal end of the handle and is provided over the hypotube. The outer sheath is configured to cover the self-expandable braided stent in a crimped state.

On rotation of the roller, the slider block is configured to translate the rotational motion of the roller assembly into forward and backward movement of the hypotube along a longitudinal axis of the delivery system, relative to the outer sheath over the inner tube.

The stent driver is configured to engage with the self-expandable braided stent and push the self-expandable braided stent out of the outer sheath to cause complete radial expansion of the self-expandable braided stent.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an isometric view of a delivery system 100 in accordance with an embodiment of the present invention.

FIG. 1A depicts a cross-sectional view of the delivery system 100 showing the internal components in accordance with an embodiment of the present invention.

FIG. 2 depicts a roller assembly 107 connected with a hypotube 105 in accordance with an embodiment of the present invention.

FIG. 2A depicts a roller 107a and a base plate 107b in accordance with an embodiment of the present invention.

FIG. 2B depicts a connecting shaft 107c in accordance with an embodiment of the present invention.

FIG. 2C depicts a slider block 107d in accordance with an embodiment of the present invention.

FIG. 2D depicts a side view of the roller assembly 107 in accordance with an embodiment of the present invention.

FIG. 3 depicts a fixing screw 109 in accordance with an embodiment of the present invention.

FIG. 4 depicts a stent driver 105d in accordance with an embodiment of the present invention.

FIG. 5 depicts a stent mounted on the delivery system 100 in crimped state in accordance with an embodiment of the present invention.

FIG. 5A depicts a magnified view of the engagement of prongs 5d2 with the stent in accordance with an embodiment of the present invention.

FIG. 6 depicts a locking pin 30 coupled to the roller 107a in accordance with an embodiment of the present invention.

FIG. 7 depicts a method for operation of the delivery system 100 for deployment of a stent in accordance with an embodiment of the present invention.

FIGS. 8A-8D depict the movement of roller 107a and its effect on the hypotube 105 for deployment of a stent in accordance with an embodiment of the present invention.

### DETAILED DESCRIPTION OF DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a delivery system for deployment of a self-expanding braided stent (hereon referred as ‘stent’) is disclosed. The delivery system of the present invention is used to deliver the stent to a target site/location. The target site of the present invention may include a lesion of a diseased peripheral, biliary or coronary vasculature.

The delivery system of the present invention includes various components such as a handle, a hypotube, a roller assembly coupled to the hypotube, a stent driver provided on the hypotube, a soft tip, an outer sheath and an inner tube. The roller assembly includes a roller, base plate, connecting shaft, slider block, etc. The aforesaid roller assembly of the present invention is very easy to use and can be operated by the use of a single hand of the operator thereby significantly reducing the efforts of the operator for deployment of the stent. The only action required by the operator is to rotate the roller while the remaining components move as a result of the rotation of the roller for deployment of the stent at the target site. Further, the use of the roller assembly of the present invention reduces the overall procedural time. Also, owing to the use of the roller assembly of the present invention, the delivery of long stents can be performed with minimum efforts and in minimum procedural time contrary to the conventional delivery systems.

The self-expandable braided stent to be delivered is initially pre-loaded over a distal end of the hypotube and retained within the outer sheath in a crimped state. The rotation of the roller by a thumb of the operator facilitates the deployment of the stent at the target site. To be precise, the rotation of the roller triggers the movement of other components of the roller assembly that cause movement of the hypotube along a longitudinal axis of the delivery system for deployment of the stent.

The operator may either rotate the roller in a clockwise or an anticlockwise direction. The roller is capable of 360-degree rotation having a first half cycle and a second half cycle of 180 degrees each. One complete rotation of the roller facilitates forward and backward movement of the hypotube along the longitudinal axis of the delivery system relative to the outer sheath, irrespective of the direction of rotation. A first half cycle of rotation of the roller causes the hypotube to move forward till a first pre-defined distance while a second half cycle of rotation causes the hypotube to move backwards covering the first pre-defined distance. Hence, one complete rotation of the roller results in zero displacement of the hypotube with respect to its original position. It should be noted that the hypotube is the only component of the delivery system of the present invention that reciprocates along the longitudinal axis on complete rotation of the roller.

As the hypotube moves forward, the hypotube ejects from the outer sheath and the stent is uncovered at least partially. The subsequent forward movements of the hypotube enable the stent driver to push the stent out of the outer sheath completely thereby causing expansion of the entire stent for deployment at the target site. The number of rotations of the roller required to deploy the stent depends upon the length of the stent to be deployed i.e., the number of rotations may be directly proportional to the length of the stent.

Therefore, owing to the presence of the roller assembly of the present invention, the delivery system of the present invention is easy to handle and operate and also, allows delivery of the stent in a controlled manner with reduced procedural time.

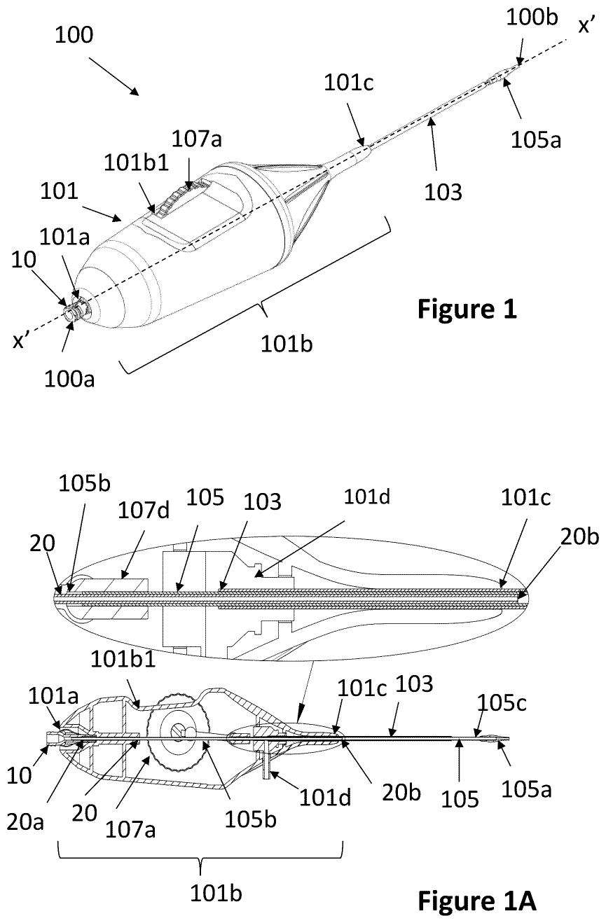

Now referring to figures, FIG. 1 depicts a delivery system 100 of the present invention. The delivery system 100 is used for deployment of at least one self-expandable braided stent (or stent) at a target site that includes without limitation, a lesion of a diseased peripheral, biliary or coronary vasculature.

The delivery system 100 of the present invention may be used for deploying stents of varied lengths. In an embodiment, the delivery system 100 is capable of deploying stents having a length ranging from 20 mm to 250 mm. Hence, in addition to small and medium sized stents, the delivery system 100 of the present invention is capable of easily delivering stents with longer lengths to the target site.

It should be noted, though not shown in FIGS. 1 and 1A, the stent is a part of the delivery system 100. An assembly of the delivery system 100 along with the stent mounted over it (in a crimped state), is packaged as is. When needed, the packaged assembly is to be utilized by an operator for further delivery and deployment of the stent (using a method described below) without making any change or amendment to the stent or the delivery system 100.

As shown in FIG. 1, the delivery system 100 extends from a proximal end 100a to a distal end 100b along a longitudinal axis x′. The delivery system 100 includes a plurality of components that are aligned along the longitudinal axis x′ of the delivery system 100. As shown in the exemplary illustration of FIG. 1, the plurality of components of the delivery system 100 includes a handle 101, an outer sheath 103 and a soft tip 105a.

As evident from FIG. 1, the handle 101 is disposed towards the proximal end 100a of the delivery system 100. The handle 101 is structured to provide proper grip to an operator for easy and controlled deployment of the stent. The handle 101 includes a pre-defined shape and dimensions that provide ergonomic benefits. Hence, the shape and dimensions i.e., length, diameter/width of the handle 101 are selected to maximize ease and comfort for the operator.

The handle 101 is made up of a pre-defined material. The pre-defined material may include, without limitation, ABS (acrylonitrile butadiene styrene) plastic, polycarbonate, HDPE (high density polyethylene), polypropylene or polyethylene, etc. In an embodiment, the handle 101 is made up of ABS.

The handle 101 forms a housing that acts as an enclosure to house various other components that function in a synchronized manner to operate and control the deployment of the stent via the delivery system 100. In an embodiment, the handle 101 is in the form of a hollow shell type of structure that includes a cavity for housing the other components as discussed below (shown in FIG. 1A).

The handle 101 includes a proximal end 101a, a distal end 101c and a central section 101b defined therebetween as seen in FIGS. 1 and 1A. In an exemplary embodiment, a luer hub 10 is disposed at the proximal end 101a of the handle 101. The luer hub 10 may be disposed in such a way that it is partially disposed within the handle 101. As shown in the exemplary embodiment of FIG. 1A, at least 10-20% of the luer hub 10 is disposed outside the handle 101 thereby defining the proximal end 100a of the delivery system 100. The luer hub 10 as conventionally known, helps in the insertion of a guidewire and to inject contrast media if required.

The luer hub 10 may be coupled to an inner tube 20 as shown in FIG. 1A. The coupling between the inner tube 20 and the luer hub 10 may be made via a pre-defined attachment means. In an exemplary embodiment, the luer hub 10 is coupled over the inner tube 20 via an ultraviolet (UV) bonding procedure.

The inner tube 20 includes a proximal end 20a and a distal end 20b as shown in FIG. 1A. The proximal end 20a of the inner tube 20 is coupled with the luer hub 10 while the distal end 20b ends near the distal end 101c along the longitudinal axis x′ of the delivery system 100.

The inner tube 20 may be made up of a pre-defined material such as stainless steel, nitinol or other metal alloys, etc. In an exemplary embodiment, the inner tube 20 is made up of stainless steel.

The distal end 101c of the handle 101 includes a hole to allow the passage of a hypotube 105. Further, the distal end 101c of the handle 101 includes a flushing port 101d as shown in FIG. 1A. The flushing port 101d is fixedly attached to the outer sheath 103. In an embodiment, along with the outer sheath 103, the flushing port 101d is attached at the distal end 101c of the handle 101 via over-molding technique (injection molding). The flushing port 101d is provided to flush the outer sheath 103 as well as to introduce contrast media between catheter and the hypotube 105.

The outer sheath 103 extends over the hypotube 105 such that the outer sheath 103 is coaxially placed over the hypotube 105. The outer sheath 103 covers the stent 200 which is mounted over the hypotube 105 in crimped state towards the distal end 100b of the delivery system 100 as shown in FIG. 5. Hence, the stent 200 is retained within the outer sheath 103 in the crimped state prior to deployment. In an embodiment, the outer sheath 103 covers the hypotube 105 in such a manner that the stent 200 is completely covered while the soft tip 105a remains uncovered by the outer sheath 103 as shown in FIGS. 1 and 5.

The outer sheath 103 may be a single-layered or a multi-layered structure. In an embodiment, the outer sheath 103 is a three-layered structure having a first layer, a second layer and a third layer. The first layer is the innermost layer that may be made up of polytetrafluoroethylene (PTFE), high density polyethylene (HDPE), etc. In an embodiment, the first layer is made up of PTFE. The second layer may be in the form of a braided layer formed by braiding flat or round wires of nitinol, stainless steel, etc. In an embodiment, the second layer is formed by braiding stainless steel round wire with one-over-one pattern. The third layer is the outermost layer which can be made up of polyether block amide, PTFE, etc. In an embodiment, the third layer is made up of polyether block amide block copolymer variation called PEBAX. It should be noted that the above details of the outer sheath 103 are only exemplary in nature and that the outer sheath 103 having different construction details can be used as per the teachings of the present invention.

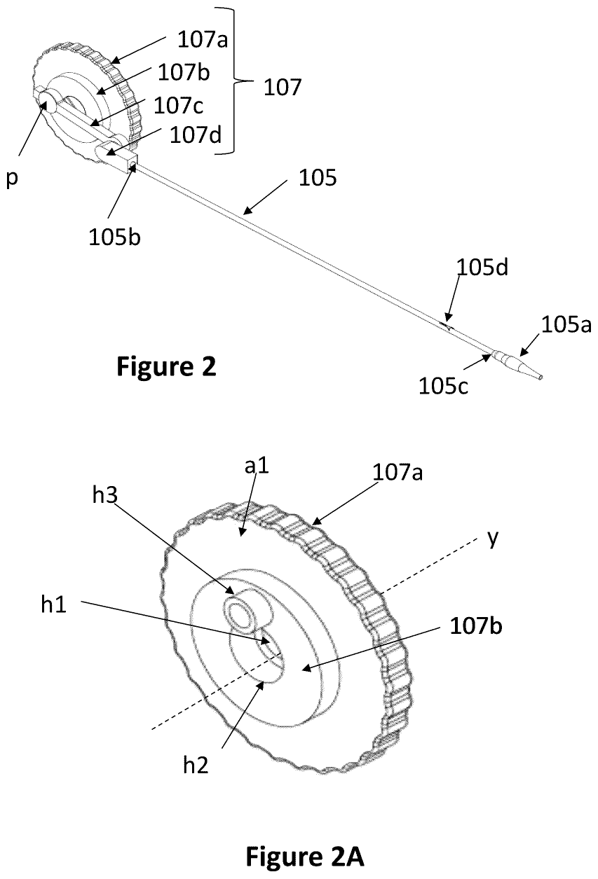

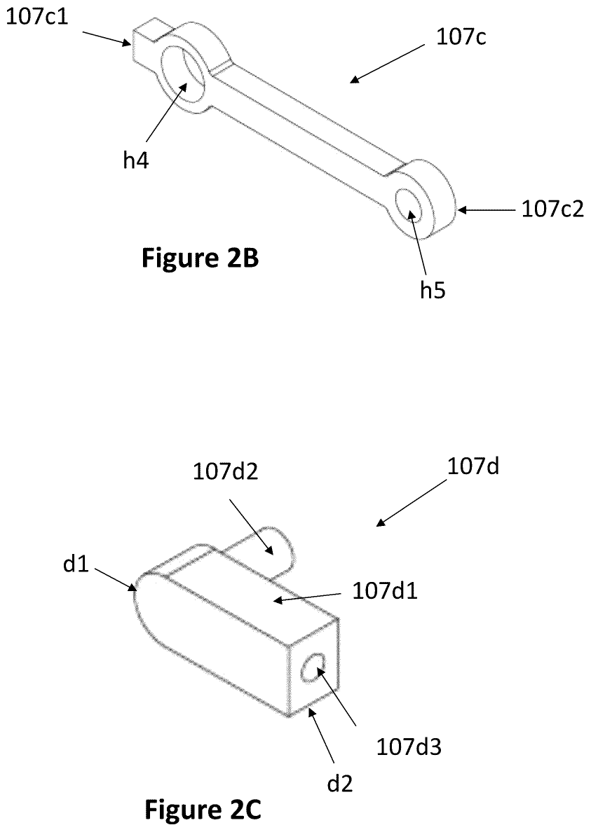

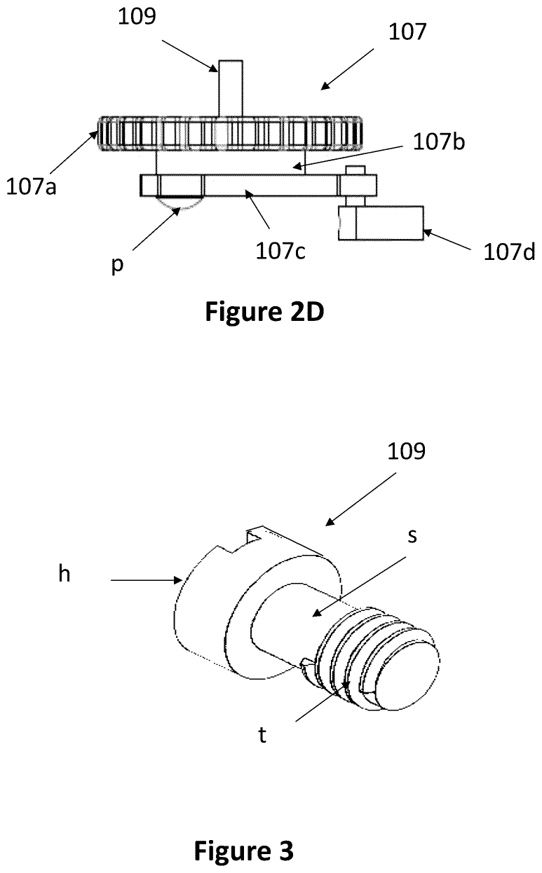

The central section 101b of the handle 101 may contain a roller assembly 107. The roller assembly 107 is provided within the housing of the handle 101 and is rotatably coupled to the handle 101. The roller assembly 107 includes a plurality of components. In an exemplary embodiment shown in FIGS. 2-2D, the roller assembly 107 includes a roller 107a, a base plate 107b, a connecting shaft 107c, and the slider block 107d. The slider block 107d may receive the hypotube 105 as shown in FIG. 2. All the components of the roller assembly 107 are assembled together to facilitate the movement of the hypotube 105 along the longitudinal axis ‘x’ of the delivery system 100 thereby resulting in easy and smooth deployment of the stent at the target site (described below in detail).

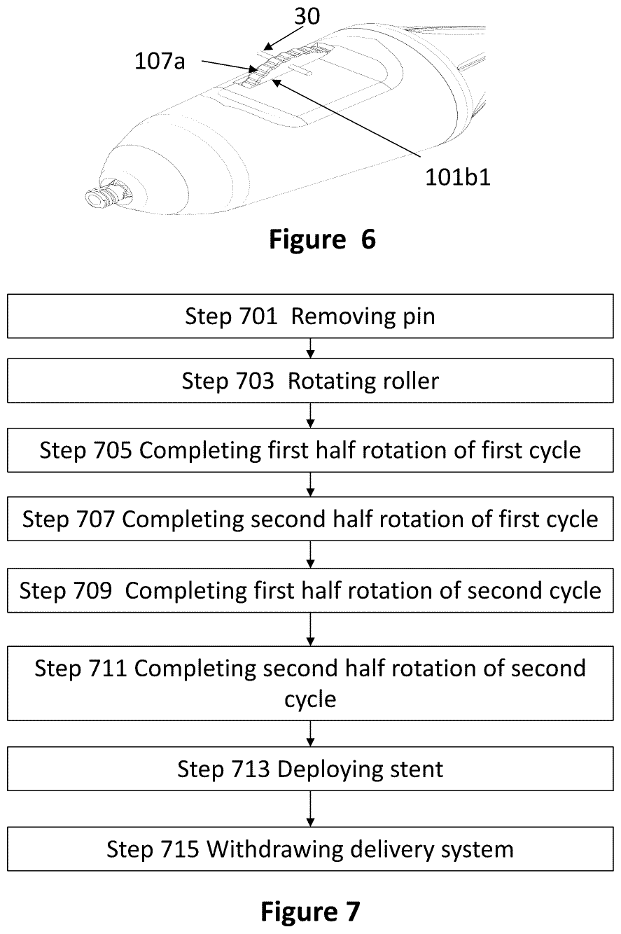

The roller 107a is capable of rotation in a clockwise as well as an anti-clockwise direction when rotated by the operator. The roller 107a is partially housed within the handle 101 in such a manner that a portion of the roller 107a protrudes out, from the periphery of the handle 101 via an opening 101b1 as shown in FIGS. 1, 1A and 6. Such a disposition allows the operator to easily access the roller 107a for rotating the same.

The roller 107a is made up of a pre-defined material. The pre-defined material may include, without limitation, ABS, polycarbonate, nylon, carbon, fiber, etc. In an exemplary embodiment, the roller 107a is made up of ABS.

The roller 107a has a pre-defined shape. The shape of the roller 107a may be, without limitation, circular, spherical, oval, polygon, scalloped circle, etc. In an exemplary embodiment, the roller 107a is circular in shape. The roller 107a includes pre-defined dimensions. In an embodiment, the roller 107a includes a thickness ranging between 4 mm to 15 mm and a diameter ranging between 25 mm to 55 mm.

Owing to the above shape and dimensions, the roller 107a includes two oppositely disposed side surfaces (a first side surface al shown in FIG. 2A and a second side surface (not shown) and a periphery. The second side surface may face an inner wall of the handle 101. The periphery of the roller 107a may be smooth or rough. In an embodiment, the periphery includes a plurality of teeth such that there is enough friction between the thumb of the operator and the roller 107a for rotating the roller 107a.

The roller 107a includes a roller hole ‘h1’ in the form of a see-through hole extending from the first side surface al to the second side surface, along an axis ‘y’ that is perpendicular to the longitudinal axis x′. The roller hole ‘h1’ may be disposed centrally or may be offset from the center of the roller 107a. In an embodiment, the roller hole ‘h1’ is centrally disposed. The roller hole ‘h1’ is of a pre-defined shape such as without limitation, circular, square, rectangle, spherical, etc. In an exemplary embodiment, the roller hole ‘h1’ is circular in shape.

The base plate 107b is disposed over the roller 107a on the first side surface al. The base plate 107b may be coupled/connected to the roller 107a via a temporary/permanent coupling means. Alternately, the base plate 107b and the roller 107a may be fabricated as an integral structure. In an exemplary embodiment, the roller 107a and the base plate 107b are fabricated integrally forming a single unit in the form of an indented structure as shown in FIG. 2A. Forming the roller 107a and the base plate 107b as a single unit prevents wear and tear thereby enhancing the durability of the delivery system 100.

The base plate 107b may be made of the same or different material as that of the roller 107a. In an embodiment, the base plate 107b and the roller 107a are both made of ABS. The base plate 107b may include a pre-defined shape such as, without limitation circular, spherical, oval, polygon, etc. In an exemplary embodiment, the base plate 107b is circular in shape.

The base plate 107b includes a pre-defined diameter and thickness. The diameter of the base plate 107b may have a diameter less than or equal to the diameter of the roller 107a. In an exemplary embodiment, the diameter of the base plate 107b is less than the diameter of the roller 107a. Further, the thickness of the base plate 107b may be same or different from the thickness of the roller 107a.

The base plate 107b is provided with a first base hole ‘h2’ and a tubular means ‘h3’ as shown in FIG. 2A. In an embodiment, the first base hole ‘h2’ is centrally disposed and the tubular means ‘h3’ is eccentrically disposed over the base plate 107b. The first base hole ‘h2’ may be disposed in such a way that the first base hole ‘h2’ coincides with the roller hole ‘h1’ in such a manner that the axis of both the holes ‘h1’ and ‘h2’ are same i.e., axis ‘y’. The first base hole ‘h2’ may include a diameter that is greater than the roller hole ‘h1’. Such dimensions help to couple the roller assembly 107 to the handle 101 (described below in detail).

The tubular means ‘h3’ may be in the form of a hollow projection that extends away from the base plate 107b as shown in FIG. 2A. The tubular means ‘h3’ may extend from the base plate 107b making an angle with respect to the base plate 107b ranging between 0 to 180 degrees. In an exemplary embodiment, the tubular means ‘h3’ is perpendicular to the base plate 107b.

The tubular means ‘h3’ may be offset from the center of the base plate 107b. In an embodiment as shown in FIG. 2A, the tubular means ‘h3’ is eccentrically disposed adjacent to the first base hole ‘h2’ towards the periphery of the base plate 107b. The aforesaid structure and disposition of the tubular means ‘h3’ helps to establish a strong connection between the connecting shaft 107c and the base plate 107b.

The dimensions as well as the shape of the tubular means ‘h3’ may be dependent upon the dimensions and shape of the connecting shaft 107c.

The connecting shaft 107c may be operatively coupled to the roller 107a via the tubular means ‘h3’ of the base plate 107b. The connecting shaft 107c is made up of a pre-defined material. The pre-defined material may include, without limitation ABS, polycarbonate, etc. In an exemplary embodiment, the connecting shaft 107c is made up of ABS.

An exemplary structure of the connecting shaft 107c is depicted in FIG. 2B which is in the form of a box ended wrench. However, other structures of the connecting shaft 107c that can facilitate similar coupling and operation are also within the scope of the present invention.

The connecting shaft 107c includes a proximal end 107c1 and a distal end 107c2. A first shaft hole ‘h4’ may be disposed towards the proximal end 107c1 while the distal end 107c2 includes a second shaft hole ‘h5’. Hence, the first shaft hole ‘h4’ and the second shaft hole ‘h5’ are spaced apart from each other.

In an embodiment, the first shaft hole ‘h4’ is configured to operatively receive the tubular means ‘h3’. Hence, the diameter of the first shaft hole ‘h4’ is slightly greater than the tubular means ‘h3’. In an embodiment, once the first shaft hole ‘h4’ is fixed over the tubular means ‘h3’, the said connection may be locked by way of a locking means. The locking means may include, without limitation, a locking pin, rivet, etc. In an exemplary embodiment, the locking means is a locking pin ‘p’ as seen in FIGS. 2 and 2D.

The second shaft hole ‘h5’ of the connecting shaft 107c may be movably coupled to the slider block 107d. Hence, the shape and dimensions of the second shaft hole ‘h5’ may be dependent upon the shape and dimensions of the slider block 107d.

An exemplary structure of the slider block 107d is shown in FIG. 2C. As evident from the exemplary depiction of FIG. 2C, the slider block 107d is in the form of a substantially rectangular shaped block 107d1 having a leg 107d2 and a cavity 107d3 extending from a proximal end ‘d1’ to a distal end ‘d2’ of the block 107d1. It should be noted that though the present invention is described by way of the aforesaid structured slider block 107d, however, other structures of the slider block 107d that result in equivalent coupling and operation of the roller assembly 107 are also within the scope of the present invention.

The leg 107d2 may extend from a proximal end d1 of the block 107d1. In an embodiment, the leg 107d2 extends away from the block 107d1 in such a manner that the leg 107d2 is perpendicular to the block 107d1 as well as the longitudinal axis x′. Alternately, the leg 107d2 may be angled with respect to the block 107d1 at an angle other than 90 degrees.

The leg 107d2 may include a pre-defined shape and dimensions. For example, the leg 107d2 is a cylindrical structure having a pre-defined length and diameter. The length of the leg 107d2 may be equivalent to the depth of the second shaft hole ‘h5’. In an embodiment, the diameter of the leg 107d2 is slightly less than the diameter of the second shaft hole ‘h5’ so that the leg 107d2 may be operatively received in the second shaft hole ‘h5’. Optionally, the coupling between the leg 107d2 and the second shaft hole ‘h5’ may be reinforced by way of a locking means such as a locking pin, rivet, etc.

As seen in FIG. 2C, the cavity 107d3 extends from the distal end d2 to the proximal end ‘d1’ of the block 107d1. The cavity 107d3 receives the inner tube 20 at the proximal end ‘d1’ of the block 107d1. Hence, the inner tube 20 that extends from the luer hub 10, passes through the slider block 107d and ends near the distal end 101c of the handle 101, thereby supporting the slider block 107d (as shown in FIG. 1A).

The cavity 107d3 also receives the hypotube 105 at the distal end ‘d2’ of the block 107d1. Hence, the shape and dimensions of the cavity 107d3 depend upon the shape and dimensions of the hypotube 105. In an exemplary embodiment, the shape of the cavity 107d3 is circular to accommodate the hypotube 105 as well as the inner tube 20, both having a circular cross-section. The diameter of the cavity 107d3 is selected to be complementary to an inner diameter of the hypotube 105 such that the hypotube 105 is fixedly received within the cavity 107d3 at the distal end d2 of the block 107d1. In an embodiment, the cavity 107d3 includes a uniform diameter which is slightly greater than the hypotube 105 so that the hypotube 105 and the inner tube 20 are co-axially received within the cavity 107d3.

Such an arrangement of the inner tube 20, the slider block 107d and the hypotube 105 allows controlled movement of the hypotube 105 along the longitudinal axis ‘x’ of the delivery system 100 over the inner tube 20, on rotation of the roller 107a. Hence, on rotation of the roller 107a, the slider block 107d is configured to translate the rotational motion of the roller assembly 107 into forward and backward movement of the hypotube 105 along the longitudinal axis ‘x’ over the inner tube 20 relative to the outer sheath 103. (described below in detail).

The above-described components of the roller assembly 107 are placed within the cavity of the handle 101 and are coupled to the handle 101 with the help of a coupling means. In an exemplary embodiment, the roller assembly 107 is rotatably coupled to the handle 101 via a fixing screw 109 as shown in FIG. 3. However, the use of other coupling means is also within the scope of the present invention.

The fixing screw 109 may be inserted through the roller 107a and the base plate 107b and fixed to an inner wall of the handle 101. The magnified view of the fixing screw 109 is shown in FIG. 3. As evident, the fixing screw 109 includes a shaft ‘s’ having a threaded tip ‘t’ and a head ‘h’. In an embodiment, the head ‘h’ is broader than the shaft ‘s’. The dimensions of the fixing screw 109 may depend upon the dimensions of the roller hole ‘h1’ and the first base hole ‘h2’. For example, the diameter of the shaft ‘s’ may be less than the diameter of the roller hole ‘h1’ and the first base hole ‘h2’. The length of the shaft ‘s’ may be more than the depth of the roller hole ‘h1’ so that the shaft ‘s’ is able to pass through the roller hole ‘h1’ and the first base hole ‘h2’. The diameter of the head ‘h’ may be more than the diameter of the roller hole ‘h1’ but slightly less than the diameter of the first base hole ‘h2’.

Hence, the shaft ‘s’ of the fixing screw 109 may first pass through the first base hole ‘h2’ and then the roller hole ‘h1’ while the head ‘h’ is fixed within the first base hole ‘h2’ owing to the selected dimensions. The threaded tip ‘t’ is then threaded to the handle 101 from the inside thereby coupling the roller assembly 107 to the handle 101.

Referring to FIGS. 1A-2 again, the hypotube 105 includes a proximal end 105b and a distal end 105c. The hypotube 105 extends along the longitudinal axis ‘x’, from the cavity 107d3 of the slider block 107d (proximal end 105b) till the distal end 100b of the delivery system 100 (distal end 105c). Hence, the proximal end 105b of the hypotube 105 is received within the cavity 107d3 of the slider block 107d. In an embodiment, the hypotube 105 is fixedly coupled in the inner diameter of the cavity 107d3 of slider block 107d. The inner diameter of the hypotube 105 is more than an outer diameter of the inner tube 20. Hence, the inner diameter of the hypotube 105 co-axially receives the inner tube 20 such that the hypotube 105 moves (reciprocates) along the longitudinal axis ‘x’ over the inner tube 20 relative to the outer sheath 103. Hence, whenever the hypotube 105 moves forward or backward, it is always supported on the inner tube 20.

The stent to be delivered is pre-loaded in a crimped state over the hypotube 105 towards the distal end 100b of the delivery system 100, covered by the outer sheath 103.

The distal end 105c of the hypotube 105 is provided with the soft tip 105a. The soft tip 105a defines the distal end 100b of the delivery system 100. The soft tip 105a facilitates atraumatic navigation of the delivery system 100 inside a patient's body. In an embodiment, the soft tip 105a is attached over a peek tube and the peek tube is further attached over the hypotube 105.

A stent driver 105d is provided towards the distal end 105c of the hypotube 105 for pushing the stent at the time of deployment. The stent driver 105d may be disposed at a pre-defined distance from the distal end 100b of the delivery system 100. In an embodiment, the pre-defined distance may range from 10 mm to 50 mm.

The stent driver 105d may be attached to a portion of a circumference of the hypotube 105 by an attachment means. The attachment means may be a temporary attachment or a permanent attachment. In an exemplary embodiment, the stent driver 105d is attached to the hypotube 105 permanently by way of welding.

The stent driver 105d is made up of a pre-defined material. The pre-defined material may include, without limitation, stainless steel, nitinol, etc. In an exemplary embodiment, the stent driver 105d is laser-cut from a nitinol tube.

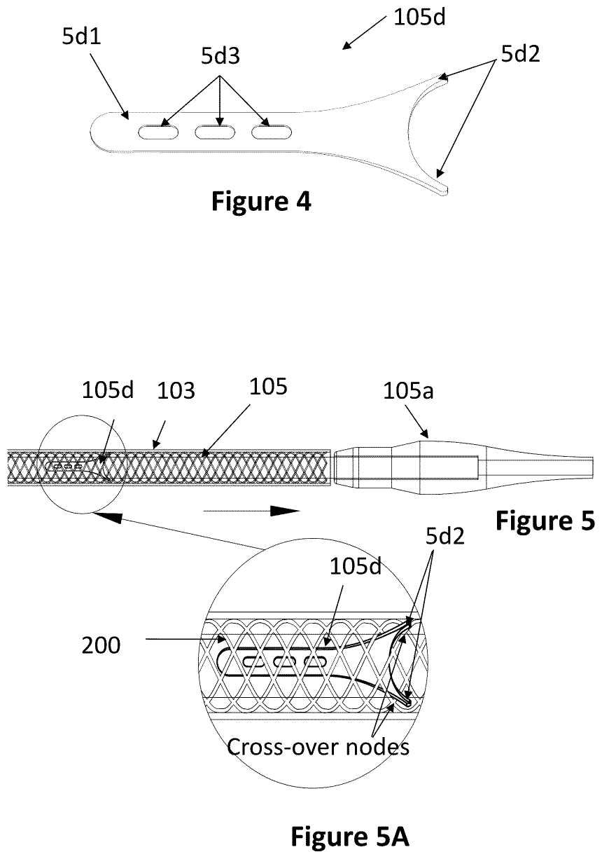

The stent driver 105d may include a pre-defined structure having a stent engagement means and a coupling portion. The stent engagement means may help in holding the stent by temporarily engaging with a set of cross-over nodes present in the stents formed by braiding. The coupling portion may help in coupling the stent driver 105d to the hypotube 105. An exemplary embodiment of the stent driver 105d is shown in FIG. 4. The stent driver 105d as illustrated in FIG. 4 is a two-prong fork type structure having a coupling portion in the form of a stem 5d1 and a stent engagement means in the form of a pair of prongs 5d2. In an exemplary embodiment, the stem 5d1 is coupled to the hypotube 105 while the prongs 5d2 have no contact with the hypotube 105. The stent driver 105d is so disposed over the hypotube 105 such that the prongs 5d2 face towards the distal end 100b of the delivery system 100. The stem 5d1 of the stent driver 105d includes a plurality of stem holes 5d3. In an embodiment, the stem 5d1 includes three stem holes 5d3. The stem holes 5d3 help at the time of welding the stent driver 105d over the hypotube 105.

The prongs 5d2 of the stent driver 105d are raised with respect to the stem 5d1 and extend away from the stem 5d1. Such a structure of the prongs 5d2 help in holding the stent by temporarily engaging with a set of cross-over nodes present in the stents formed by braiding (explained below in detail).

The above-described delivery system 100 is capable of easily deploying the stent at the target site, with the help of the roller assembly 107.

As mentioned above and shown in FIG. 5, the delivery system 100 loaded with the crimped stent, is packaged as an assembly. The outer sheath 103 covers the hypotube 105 such that the stent remains completely covered by the outer sheath 103 and only the soft tip 105a is exposed as shown in FIG. 5.

Further, as seen in the exemplary embodiment of FIG. 5A, a stent 200 is mounted in such a way that the stent driver 105d is in contact with the stent 200 at a pre-defined location. In an embodiment, the prongs 5d2 of the stent driver 105d hold on to the stent 200 by engaging with respective cross-over nodes disposed at a proximal portion (disposed towards a stent's proximal end) of the stent 200 as shown in FIG. 5A more clearly.

It should be noted that in order to prevent the accidental rotation of the roller 107a when the delivery system 100 is not in use i.e., when the delivery system 100 is packaged or prior to deployment, the roller 107a may be locked. In an embodiment, the roller 107a may be locked by way of a lock pin 30 as shown in FIG. 6. The lock pin 30 may be removably attached to the roller 107a.

The lock pin 30 may be in the form of a cylindrical rod as shown in FIG. 6. However, other structural embodiments of the lock pin 30 having equivalent functionality are also within the scope of the present invention. The roller 107a in this embodiment may include a see-through lock hole to allow the passage of the lock pin 30 through the roller 107a. The see-through hole may be shaped and dimensioned in accordance with the shape and dimensions of the lock pin 30.

Owing to the presence of the lock pin 30, the rotation of the roller 107a is restricted.

The aforesaid delivery system 100 operates in a pre-defined manner to deploy the stent 200. FIG. 7 depicts an exemplary embodiment of the mechanism of operation of the delivery system 100 and the steps followed to deploy the stent 200 at the target site.

At the time of the deployment procedure, the stent 200 pre-loaded over the hypotube 105, is navigated within the patient's body to arrive at the target site. Once the target site is reached, the lock pin 30 is removed at step 701 such that the roller 107a can be freely rotated.

At step 703, the roller 107a is rotated in a clockwise or an anti-clockwise direction by the operator. It should be noted that, both, the clockwise and the anti-clockwise rotation of roller 107a yield same result. Hence, irrespective of the direction of rotation, the mechanism of operation of the delivery system 100 to deploy the stent 200 remains the same.

As mentioned above, the roller 107a is capable of 360 degrees rotation. In a complete cycle of 360 degrees rotation, the hypotube 105 first moves forward followed by backward movement along the longitudinal axis of the delivery system 100, irrespective of the direction of rotation of the roller 107a. Each cycle of 360-degree rotation (cycle of rotation) includes two half cycles i.e., a first half cycle of rotation and a second half cycle of rotation, of 180 degrees each (in clockwise or anti-clockwise direction).

It should be noted that, in the first half cycle of rotation, the hypotube 105 is configured to move forward and the stent engagement means is configured to engage with the set of cross-over nodes of the stent 200. The set of cross-over nodes in each rotation lies towards the proximal end 100a of the delivery system 100 compared to a previous rotation. Hence, in every first half cycle of rotation, the hypotube 105 moves forward to facilitate the engagement of the stent engagement means with a new set of cross-over nodes that lie nearer to the proximal end 100a relative to a previous set of cross-over nodes.

In the second half cycle of rotation, the hypotube 105 moves backwards and the stent engagement means is disengaged with the set of cross-over nodes of the stent 200.

The number of cycles of rotation vary depending upon the length of the stent 200. For explanation purposes, the below method is described by way of two 360-degree rotations i.e., a first cycle of rotation of 360-degree and a second cycle of rotation of 360-degree.

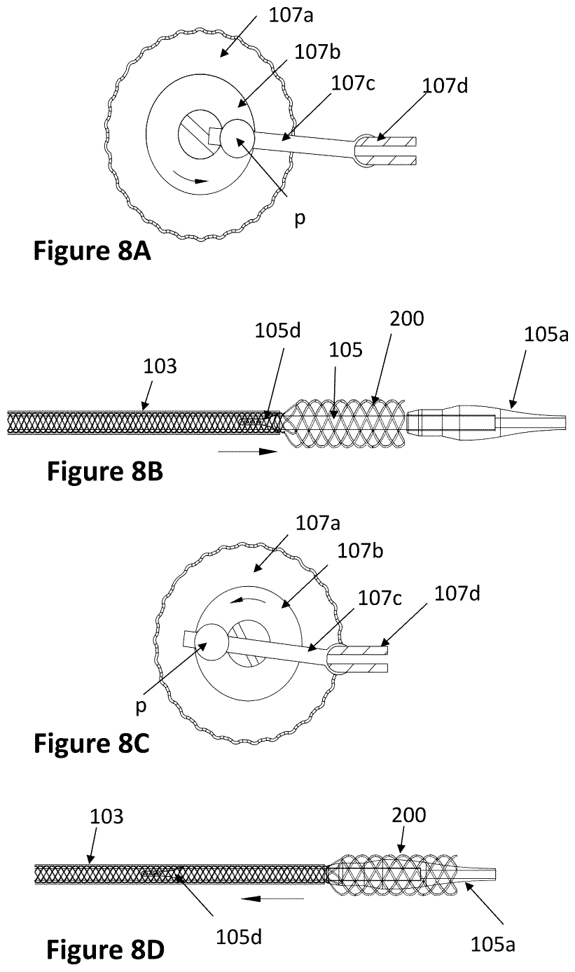

At step 705, upon completion of a first half cycle (180-degree rotation) of the first cycle of rotation (360-degree rotation), of the roller 107a, a simultaneous 180-degree rotation of the connecting shaft 107c is caused as the roller 107a and the connecting shaft 107c are operatively coupled to each other as shown in FIG. 8A. In an embodiment, the connecting shaft 107c rotates in a same direction as that of the roller 107a.

The rotational motion of the roller assembly 107 is translated into movement of the hypotube 105 along the longitudinal axis ‘x’ by way of the slider block 107d. At this stage, the hypotube 105 moves forward with respect to the outer sheath 103 as shown in FIG. 8B. It should be noted that during the forward movement of the hypotube 105, the prongs 5d2 remain engaged with the set of cross-over nodes of the stent 200.

The forward movement of the hypotube 105 uncovers at least a distal portion of the stent 200 as shown in FIG. 8B. The portion of the stent 200 that is uncovered by the outer sheath 103, expands instantly while the remaining portion (if any) is still in crimped state covered by the outer sheath 103. The same is seen in FIG. 8B.

As soon as the stent 200 is expanded, the prongs 5d2 of the stent driver 105d disengage with the set of cross-over nodes of the stent 200.

At step 707, the roller 107a is further rotated to complete a second half cycle (180-degree rotation) of the first cycle of rotation. The rotation of the roller 107a causes simultaneous 180-degree rotation of the connecting shaft 107c as the roller 107a is operatively coupled to the connecting shaft 107c as shown in FIG. 8C. The rotational motion of the roller assembly 107 is translated into the movement of the hypotube 105 along the longitudinal axis ‘x’ by way of the slider block 107d. At this stage, the hypotube 105 moves backwards with respect to the outer sheath 103. In an embodiment, the hypotube 105 moves backwards covering the first distance. Hence, one complete cycle of rotation results in zero displacement of the hypotube 105 with respect to its original position.

It should be noted that, given the prongs 5d2 are already disengaged with the stent 200, the stent 200 retains its position as attained at the end of step 705 and does not move backwards with the hypotube 105 on the backward movement of the hypotube 105 as shown in FIG. 8D.

At step 709, the roller 107a is rotated to complete a first half cycle (180-degree rotation) of the second cycle of rotation (360-degree rotation). At this step, the hypotube 105 moves forward with respect to the outer sheath 103. During the forward movement of the hypotube 105 and prior to covering the first distance, the stent driver 105d contacts the proximal portion of the stent 200 that is still in crimped state covered by the outer sheath 103. At this stage, the prongs 5d2 engage with a new set of cross-over nodes present on the proximal portion of the stent 200 that is in crimped state.

It should be noted that every time the hypotube 105 moves forward, the prongs 5d2 engage with a new set of cross-over nodes. Each new set of cross-over nodes is disposed more proximal to the proximal end of the stent 200 (and also the proximal end 100a of the delivery system 100) than the preceding set of cross-over nodes. The below table provides an exemplary table to illustrate the aforesaid:

| State of hypotube 105 | Engagement of prongs 5d2 |
|---|---|
| Initial (as packaged) | 3^{rd} set of cross-over nodes from the proximal end |
| | of the stent |
| 1^{st} forward movement (first half cycle | 3^{rd} set of cross-over nodes from the proximal end |
| of first cycle of rotation) | of the stent |
| 1^{st} backward movement (second half | Disengagement of the prongs 5d2 with the 3^{rd} set |
| cycle of first cycle of rotation) | of cross-over nodes from the proximal end of the |
| | stent |
| 2^{nd} forward movement (first half cycle | 1^{st} set of cross-over nodes from the proximal end |
| of second cycle of rotation) | of the stent leading to complete radial expansion |
| 2^{nd} backward movement (second half | Disengagement of the prongs 5d2 with the 1^{st} set |
| cycle of second cycle of rotation) | of cross-over nodes from the proximal end of the |
| | stent |

The stent driver 105d, post engagement of the prongs 5d2 and the cross-over nodes further pushes the stent 200 for complete expansion, till the hypotube 105 covers the first distance (i.e., till the completion of the first half cycle of the second cycle of rotation).

At step 711, the roller 107a is rotated to complete a second half cycle (180-degree rotation) of the second cycle of rotation. At this step, the hypotube 105 moves backwards with respect to the outer sheath 103. As the prongs 5d2 disengage with the stent 200, the hypotube 105 moves backwards without affecting the stent 200 similar to step 707.

It should be noted that depending upon the length of the stent 200, the roller 107a is rotated in the same manner multiple times (clockwise/anti-clockwise direction) as elaborated above in steps 703-711 to push the stent 200 out of the outer sheath 103 till the stent 200 is completely expanded at step 713. For example, the number cycles of rotations required for short stents (20 mm to 40 mm) may be less than the medium length stents (60 mm to 80 mm) and long stents (100 mm to 250 mm). In an exemplary embodiment, a stent 200 having a length of 150 mm requires 38 cycles of rotation for its deployment.

At step 713, the stent 200 expands to its deployed diameter at the target site and the delivery system 100 is subsequently withdrawn at step 715.

The deployment of the stent 200 using the delivery system 100 via the roller assembly 107 is simple and error free. As described above, the rotation of the roller 107a causes the movement of the connecting shaft 107c automatically which then results in movement of the hypotube 105 along the longitudinal axis ‘x’ to deploy the stent 200 at the target site.

The delivery system 100 of the present invention as described above is associated with the following advantages:
1. Single-handed operation: The delivery system 100 of the present invention can be operated using a single hand unlike the conventional delivery systems that require simultaneous usage of both the hands of the operator. The single-handed operation of the delivery system 100 eliminates all possible errors caused due to synchronization of both the hands by the operator, thereby facilitating error-free delivery and simple deployment of the stent 200. The delivery system 100 of the present invention significantly reduces the efforts of the operator as evident from the foregoing description.
2. Procedural time: Further, the use of the roller assembly 107 of the present invention reduces the overall procedural time. Also, owing to the use of the roller assembly 107 of the present invention, the delivery of long stents can be performed with minimum efforts and in minimum procedural time contrary to the conventional delivery systems.
3. Application of mind: The roller assembly 107 is very user friendly and does not require specialized application of mind as the only effort required by the operator is the rotation of the roller 107a. Such mechanism of the present invention further reduces errors and chances of inaccurate deployment of the stent 200.
4. Training: Given that the operation of the delivery system 100 only requires rotation of the roller 107a without any consideration or special care, no dedicated preparation or training is required to be undertaken by the operator (physician). The operator can straightaway use the delivery system 100 for deployment of the stent 200.
Therefore, the delivery system 100 of the present invention is easy to handle and operate and allows delivery of the stent in a controlled manner.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

1. A delivery system (100) for delivering a self-expandable braided stent (200), the delivery system (100) comprising:
a handle (101) forming a housing, the handle (101) having a distal end (101c);
a roller assembly (107) provided within the housing and rotatably coupled to the handle (101), the roller assembly (107) including:
a roller (107a) capable of rotation, the roller (107a) having a roller hole (‘h1’);
a base plate (107b) connected to or integral with the roller (107a), the base plate (107b) having a first base hole (‘h2’) and a tubular means (‘h3’), the first base hole (‘h2’) coincides with the roller hole (‘h1’);
a connecting shaft (107c) having a first shaft hole (‘h4’) and a second shaft hole (‘h5’), the first shaft hole (‘h4’) operatively receives the tubular means (‘h3’),
a slider block (107d) including a block (107d1) having a cavity (107d3) extending from a proximal end (‘d1’) to a distal end (‘d2’) of the block (107d1) and a leg (107d2) extending away from the proximal end (‘d1’) of the block (107d1), the leg (107d2) being operatively received in the second shaft hole (‘h5’);
an inner tube (20) extending from a luer hub (10) through the cavity (107d3) from the proximal end (‘d1’) and ending near the distal end (101c) along a longitudinal axis (‘x’) of a delivery system (100);
a hypotube (105) being fixedly received within the cavity (107d3) from the distal end (d2) of the block (107d1) and co-axially receives the inner tube (20), the hypotube (105) being mounted with a self-expandable braided stent (200) in a crimped state towards a distal end (100b) of the delivery system (100); and
an outer sheath (103) provided over the hypotube (105) to cover the self-expandable braided stent (200),
wherein on rotation of the roller (107a), the slider block (107d) is configured to translate rotational motion of the roller assembly (107) into forward and backward movement of the hypotube (105) along the longitudinal axis ‘x’ over the inner tube (20) relative to the outer sheath (103).

2. The delivery system (100) for delivering the self-expandable braided stent (200) as claimed in claim 1, wherein the self-expandable braided stent (200) includes a length ranging from 20 mm to 250 mm.

3. The delivery system (100) for delivering the self-expandable braided stent (200) as claimed in claim 1, wherein the roller (107a) includes a plurality of teeth over its periphery.

4. The delivery system (100) for delivering the self-expandable braided stent (200) as claimed in claim 1, wherein the distal end (100b) is provided with a soft tip (105a).

5. The delivery system (100) for delivering the self-expandable braided stent (200) as claimed in claim 1, wherein the roller assembly (107) is rotatably coupled to an inner wall of the handle (101) via a fixing screw (109) that passes through the roller hole ‘h1’ and the first base hole ‘h2’.

6. The delivery system (100) for delivering the self-expandable braided stent (200) as claimed in claim 1, wherein the roller (107a) is locked by a removable lock pin (30) that restricts accidental rotation of the roller (107a) when the delivery system (100) is not in use.

7. The delivery system (100) for delivering the self-expandable braided stent (200) as claimed in claim 1, wherein the cavity (107d3) includes a uniform diameter complementary to an inner diameter of the hypotube (105).

8. The delivery system (100) for delivering the self-expandable braided stent (200) as claimed in claim 1, wherein the hypotube (105) is provided with a stent driver (105d) that includes a stent engagement means for temporarily engaging with a set of cross-over nodes of the self-expandable braided stent (200).

9. The delivery system (100) for delivering the self-expandable braided stent (200) as claimed in claim 1, wherein the roller (107a) is rotated multiple times, either in a clockwise or an anti-clockwise direction, depending upon a length of the self-expandable braided stent (200) to push the self-expandable braided stent (200) out of the outer sheath (103).

10. The delivery system (100) for delivering the self-expandable braided stent (200) as claimed in claim 1, wherein each rotation of the roller (107a) includes a first half cycle of rotation and a second half cycle of rotation of 180-degrees each in a clockwise or an anti-clockwise direction.

11. The delivery system (100) for delivering the self-expandable braided stent (200) as claimed in claim 10, wherein in the first half cycle of rotation, the hypotube (105) is configured to move forward and a stent engagement means is configured to engage with a set of cross-over nodes of the self-expandable braided stent (200) such that the set of cross-over nodes engaged in each rotation is nearer to a proximal end (100a) of the delivery system (100) relative to the set of cross-over nodes in a previous rotation.

12. The delivery system (100) for delivering the self-expandable braided stent (200) as claimed in claim 10 wherein, in the second half cycle of rotation, the hypotube (105) is configured to move backwards and a stent engagement means is configured to disengage with a set of cross-over nodes of the self-expandable braided stent (200).
